(19) United States
(12) Patent Application Publication
(10) Pub. No.: US 2022/0175348 A1
(43) Pub. Date: Jun. 9, 2022

(54) ULTRASONIC DIAGNOSTIC APPARATUS

(71) Applicant: FUJIFILM Healthcare Corporation, Chiba (JP)

(72) Inventor: Eiji KASAHARA, Chiba (JP)

(21) Appl. No.: 17/457,275

(22) Filed: Dec. 2, 2021

(30) Foreign Application Priority Data
Dec. 9, 2020 (JP) 2020-204043

(51) Int. Cl. A61B 8/00; A61B 8/08

(52) U.S. Cl. CPC A61B 8/461; A61B 8/52; A61B 8/54

(57) ABSTRACT

A camera is disposed on a display. A camera image is analyzed to determine a representative coordinate of an operator. The orientation of the display is controlled such that the representative coordinate is within a target area of the camera image. The position and attitude of the display may be controlled, based on the camera image, to avoid illumination reflection.

### CROSS REFERENCE TO RELATED APPLICATION

This application claims priority to Japanese Patent Application No. 2020-204043 filed on Dec. 9, 2020, which is incorporated herein by reference in its entirety including the specification, claims, drawings, and abstract.

### TECHNICAL FIELD

The present disclosure relates to an ultrasonic diagnostic apparatus, and more particularly to control of the orientation of a display.

### BACKGROUND

Ultrasonic diagnostic apparatuses are medical apparatuses that form an ultrasound image based on reception signals acquired by transmitting ultrasound waves to a subject (living body) and receiving reflection waves from within the subject. Various types of ultrasonic diagnostic apparatuses are currently in practical use. Among these types, a cart-type ultrasonic diagnostic apparatus includes an apparatus body that supports an operation panel via an elevator mechanism. An articulated support mechanism is disposed on a seat located at the back of the operation panel to hold a display. The position and attitude of the display may be changed as desired by an operator.

JP 2007-21088 A (Document 1) discloses an ultrasonic diagnostic apparatus configured to automatically adjust a display to a preset position.

### SUMMARY

The position of the operator's head is continuously changing during ultrasonic inspection or during probe operation. To view an ultrasound image displayed on the display more easily and clearly, the operator may wish to adjust the orientation of the display. It is, however, usually difficult for the operator to change the orientation of the display during probe operation, because the display is out of reach of the operator, because both hands of the operator are occupied due to operation of the probe and the operation panel, because the inspection time needs to be as short as possible, and other reasons.

An embodiment of the disclosure is therefore directed toward assisting operator's observation of an ultrasound image or helping the operator view an ultrasound image easily regardless of the position of the operator's head.

In an aspect of the disclosure, an ultrasonic diagnostic apparatus includes a display configured to display an ultrasound image, a camera disposed on the display or adjacent to the display to capture an image of a front space including an operator and form a camera image, a support mechanism supporting the display and including a driving source configured to change at least one of a position and attitude of the display, and a controller configured to control the driving source based on an operator image included in the camera image.

### BRIEF DESCRIPTION OF DRAWINGS

An embodiment of the present disclosure will be described based on the following figures, wherein:

FIG. 1 is a block diagram illustrating an ultrasonic diagnostic apparatus according to an embodiment;

FIG. 2 is a side view illustrating an ultrasonic diagnostic apparatus according to an embodiment;

FIG. 3 illustrates a control algorithm according to an embodiment;

FIG. 4 illustrates an example of tracking control;

FIG. 5 illustrates a tracking control method;

FIG. 6 illustrates reflection of an illumination image;

FIG. 7 illustrates avoidance of reflection of an illumination image;

FIG. 8 illustrates a reflection avoiding method; and

FIG. 9 illustrates tracking control and avoidance control.

### DESCRIPTION OF EMBODIMENTS

An embodiment will be described by reference to the drawings.

### (1) Summary of Embodiment

An ultrasonic diagnostic apparatus according to an embodiment includes a display, a camera, a support mechanism, and a control unit. The display displays an ultrasound image. The camera is located on the display or near the display to capture an image of a front space including an operator and produce a camera image. The support mechanism is configured to support the display, and includes a driving source that changes at least one of the position and attitude of the display. The control unit controls the driving source based on an operator image included in the camera image. The control unit may also be referred to as a controller.

The above configuration automatically changes at least one of the position and attitude of the display in accordance with the position of the operator within the front space. For example, the orientation of the display is adaptively changed such that the display faces the operator's head or the operator's face to thereby facilitate observation of an ultrasound image.

The above configuration effectively works when the operator cannot change the orientation of the display manually with both hands of the operator being occupied, when the operator's hand that is not holding the probe cannot reach the display, or when there is insufficient time for adjusting the orientation of the display because the inspection time needs to be shortened, for example.

In an embodiment, the camera is fixed to the display, and the imaging field of view of the camera and the observation field of view of the display overlap with each other. As the display and the camera are integrated, this configuration reflects a spatial relationship between the display and the operator in the location of an operator image within the camera image. The camera may be embedded in the display or may be externally fixed to the display. In either configuration, the spatial relationship between the camera and the display is fixed to capture an image of the space in front of the display. The imaging field of view corresponds to a range in which imaging can be performed, and the observation field of view corresponds to a range in which observation can be performed. In practice, the observation field of view suitable for image diagnosis is not very wide.

In an embodiment, the control unit determines a representative position in the operator image within the camera image, and changes at least one of the position and attitude of the display based on the representative position. The representative position may be a specific position within a head image, a specific position within a face image, positions of two eyes, or an intermediate position between the two eyes, for example.

In an embodiment, the representative position is determined based on the head image within the operator image. The control unit changes at least one of the position and attitude of the display such that the representative position corresponds to or comes close to a target position within the camera image. This configuration enables the display screen to continuously face the head or face of the operator. The target position may be a point, a line, or a region.

In an embodiment, the control unit determines, based on the camera image, whether the operator is facing the display, and, in response to determination of the operator facing the display, controls the driving source. This configuration avoids unnecessary change of the position or attitude of the display. The control unit may automatically stop controlling the driving source in a freeze state or while a probe is not in contact with a surface of a living body.

In an embodiment, the control unit determines, in accordance with a designated response condition, a moving velocity of the display in changing at least one of the position or attitude of the display. A motion to change the position and attitude of the display that is too fast or too slow would put stress on the operator. The above configuration eliminates or alleviates such a stress.

In an embodiment, the control unit determines whether or not illumination reflection on an image displayed on the display is present based on an illumination image included in the camera image, and, in response to determination of presence of the illumination reflection, changes at least one of the position and attitude of the display to thereby reduce or eliminate the illumination reflection. This configuration resolves or lessens an issue caused by the illumination reflection (difficulty in image observation).

In an embodiment, the control unit determines presence of the illumination reflection when a contour or representative coordinate of the illumination image lies within a determination region within the camera image, and changes at least one of the position and attitude of the display such that the contour or representative coordinate of the illumination image is outside of the determination region. At this time, the height and tilt angle of the display may be changed simultaneously, for example.

The ultrasonic diagnostic apparatus according to an embodiment includes tracking control for continuously directing the display to the operator and avoidance control for automatically avoiding or reducing illumination reflection. These control operations are basically independent from each other, but may be executed in combination. Both control operations are techniques for assisting the operator based on a camera image.

### (2) Details of Embodiment

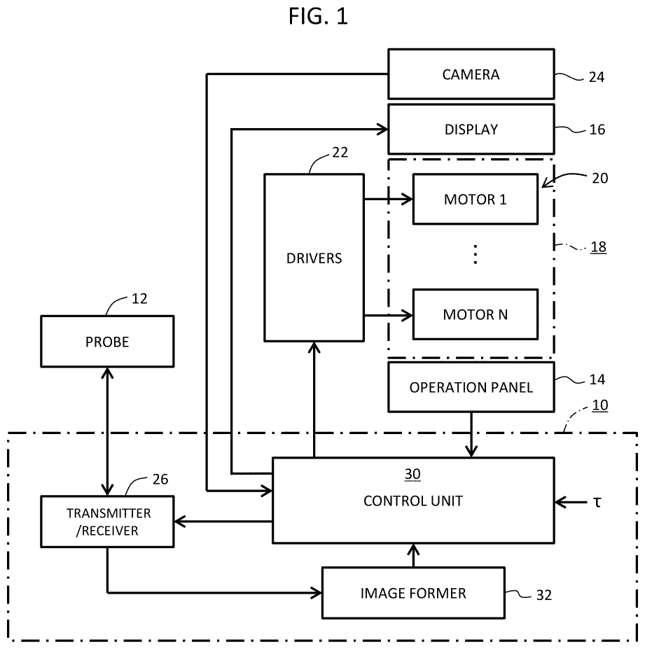

FIG. 1 is a block diagram illustrating an example configuration of an ultrasonic diagnostic apparatus according to an embodiment. The ultrasonic diagnostic apparatus is a medical apparatus installed in hospitals and other medical institutions. The ultrasonic diagnostic apparatus transmits ultrasound waves to a subject (living body) and receives reflection waves from within the subject, and, based on information acquired from the reflection waves, forms an ultrasound image representing a tissue within the subject. The ultrasonic diagnostic apparatus according to the embodiment is a cart-type ultrasonic diagnostic apparatus.

The ultrasonic diagnostic apparatus includes a body (ultrasonic diagnostic apparatus body) 10. A probe (ultrasonic probe) 12 is detachably coupled to the body 10. The body 10 supports an operation panel 14 via an elevator mechanism. A seat is disposed at the back of the operation panel 14 to place a support mechanism 18 thereon. The support mechanism 18 is an articulated mechanism, as will be described below, and holds a display 16. The display 16 is a flat panel display and specifically includes an LCD, an organic EL display device, and other displays.

The support mechanism 18 includes a plurality of motors or a plurality of actuators as a driving source 20 to drive each movable portion of the support mechanism 18. A plurality of driving signals are supplied in parallel from drivers 22 to the plurality of motors. The drivers 22 are disposed outside or inside the support mechanism 18. The drivers 22 may be disposed within the body 10.

The operation panel 14 is an input device having a plurality of switches, a plurality of buttons, a trackball, and a keyboard, for example. During ultrasonic inspection, the operator typically holds the probe 12 with one hand and operates the operation panel 14 with the other hand.

A camera 24 is secured to an upper part of the display 16. In this embodiment, the camera 24 is embedded in an upper part of the display 16. As will be described below, the camera 24 captures an image of a space in front of the display 16 or a space including the operator, as a moving image. The observation field of view of the display 16 and the imaging field of view of the camera 24 overlap with each other. The observation field of view refers to a spatial range of the display 16 in which observation can be performed and the imaging field of view refers to a spatial range in which image capturing can be performed. In practice, the observation field of view is a range in which image diagnosis can be performed and is not very wide. The camera 24 is a monochrome camera or a color camera. A plurality of cameras may be disposed. The camera 24 may be fixedly disposed outside the display 16.

The probe 12 is composed of a probe head, a cable, and a connector. The probe 12 includes, within the probe head, a transducer element array formed of a plurality of transducer elements arranged linearly or in an arc shape. Ultrasound waves are transmitted from the transducer element array into the subject and reflection waves from within the subject are received by the transducer element array. More specifically, the transducer element array forms ultrasound beams (transmitting beams and received beams), which are electronically scanned to form a scan plane (beam scan plane). Known electronic scanning methods include an electronic sector scanning method and an electronic linear scanning method, for example. The probe head is a primary portion and is to be held by the operator. In place of a one-dimensional transducer element array, a two-dimensional transducer element array may be disposed.

A transmission/reception unit 26 is an electronic circuit that functions as a transmitting beam former and a received beam former. In transmission, the transmission/reception unit 26 provides a plurality of transmitting signals to the transducer element array in parallel, thereby forming transmitting beams. In reception, upon receiving reflection waves from within the living body, the transducer element array outputs a plurality of received signals to the transmission/reception unit 26. The transmission/reception unit 26 then applies phase alignment and summing (delay and summing) to the plurality of received signals, thereby generating received beam data. Typically, a single scan of the ultrasound beam generates a single set of received frame data. One set of received frame data is composed of a plurality of received beam data items arranged in the electronic scanning direction. One received beam data item is composed of a plurality of echo data items arranged in the depth direction. The ultrasound beams are electronically scanned in a repeated manner and a plurality of received frame data items are generated repeatedly and form received frame data sequences.

An image former 32 generates a tomographic image data sequence based on the received frame data sequence. Specifically, the image former 32 includes a digital scan converter (DSC) that is a special processor having a coordinate conversion function, a pixel interpolation function, a frame rate conversion function, and other functions. In the illustrated example configuration, the tomographic image data sequence is transmitted from the image former 32 to a control unit 30.

The control unit 30 is composed of a processor that executes a program. Specifically, the control unit 30 is composed of a CPU. The control unit 30 has, in addition to a function to control operations of components forming the ultrasonic diagnostic apparatus, an image processing function and a display processing function, for example. The control unit 30 according to the embodiment executes tracking control and avoidance control simultaneously or selectively during the ultrasonic inspection. Under the tracking control, the screen of the display 16 is kept facing the operator's face. Under the avoidance control, reflection of illumination on the screen of the display 16, when viewed from the operator, is avoided. In practice, the position and attitude of the display are optimized under control of the motion of the driving source 20.

The camera 24 transmits camera image data to the control unit 30, and the control unit 30 transmits the ultrasound image data to the display 16. The display 16 then displays an ultrasound image. The camera image captured by the camera 24 may be displayed on the display 16 as required.

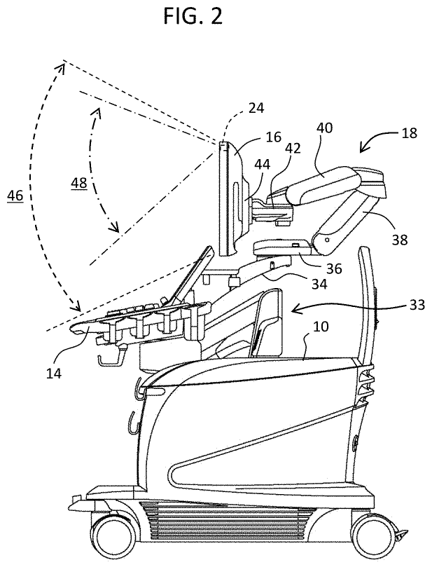

FIG. 2 illustrates the outer appearance of the ultrasonic diagnostic apparatus. The body 10 has a box shape and has four casters at the bottom. An elevator mechanism 33 is disposed between the body 10 and the operation panel 14, and the body 10 supports the operation panel 14 via the elevator mechanism 33. A seat 34 is disposed at the back of the operation panel 14, and the support mechanism 18 is disposed on the seat 34.

The support mechanism 18 is an arm mechanism serving as an articulated mechanism, and includes a plurality of motors as a driving source. The support mechanism 18 supports the display 16. The shape of the support mechanism 18, for example, is changed to thereby change the position and attitude of the display 16.

In the illustrated example configuration, the support mechanism 18 includes a first arm 36, a second arm 38, a third arm 40, a fourth arm 42, and a tilt mechanism 44, for example. The first arm 36 turns with respect to the seat 34. The second arm 38 includes a parallel link and tilts with respect to the first arm 36. The second arm 38 has an upper end that is coupled with a proximal end of the third arm 40. The third arm 40 turns with respect to the second arm 38. The third arm 40 also includes a parallel link and tilts with respect to the second arm 38. The fourth arm 42 turns with respect to the third arm 40. The tilt mechanism 44 allows the display 16 to rotate about a horizontal rotation axis. The support mechanism 18 that is illustrated is only one example, and various types of mechanisms that automatically move may be employed for the support mechanism 18.

The display 16 has a screen on the front side, and includes the camera 24 embedded in the center of the upper portion. The observation field of view 46 of the display 16 and the imaging field of view 48 of the camera 24 overlap with each other. The spatial relationship between the display 16 and the operator can be specified through the camera image. The position of the operator, and particularly the position of the operator's head, can also be specified through analysis of the camera image. The observation field of view 46 and the imaging field of view 48 also overlap when they are viewed from above, and particularly, their center axes correspond to each other.

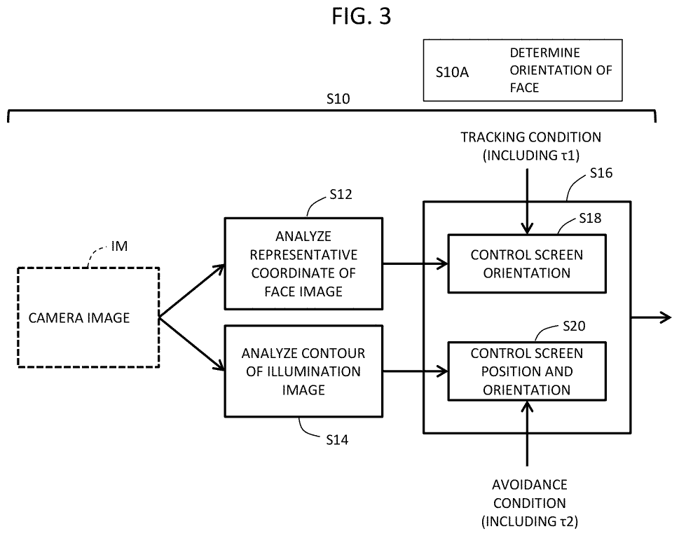

FIG. 3 illustrates a control algorithm. In step S10, whether the tracking control and/or the avoidance control is to be executed is determined. A user may select the control mode to be executed. The tracking control may be executed only during transmission and reception of ultrasound.

For example, the orientation of the operator's face is determined based on an operator image in a camera image as indicated in step S10A. More specifically, whether or not the operator is facing the display is determined. Only when the operator is facing the display, execution of various steps after steps S12 and S14 may be allowed. Stated conversely, when the operator is not facing the display, execution of the various steps after steps S12 and S14 may be prohibited. Control of the support mechanism may be temporarily stopped during a freeze state or while the probe is left to air.

In response to an instruction to execute the tracking control, the processes in steps S12 and S18 are to be executed. In image analysis in step S12, a head image or a face image is specified based on a camera image IM, and a representative coordinate is specified based on the head image. In step S18, in accordance with a designated tracking condition, the orientation of the display screen is controlled based on the representative coordinate of the operator. For example, in response to the determination that the representative coordinate is outside of a target area within the camera image, the orientation of the display screen and simultaneously, the orientation of the camera, are adaptively controlled such that the representative coordinate is within the target area. This control allows the screen to keep facing the operator. During the ultrasonic inspection, the orientation of the screen is thus optimized automatically in accordance with a change in the position of the operator's face.

The tracking condition includes a time constant τ1 as a response condition. The smaller the time constant τ1, the faster the tracking. The tracking condition may include the size and location of the target area.

In response to an instruction to execute the avoidance control, the processes in steps S14 and S20 are to be executed. In image processing in step S14, whether or not illumination reflection is present is determined based on the camera image, and, in response to determination of presence of illumination reflection, a contour of an illumination image is extracted. In step S20, the position and attitude of the screen are changed in accordance with an avoidance condition such that a contour image of the illumination image is removed outside of a determination area in the camera image. For example, the display is raised and is simultaneously oriented downward so that the illumination image is removed from the screen viewed from the operator. In place of the contour of the illumination image, a representative coordinate of the illumination image may be used.

The avoidance condition includes a time constant τ2 as a response condition. The smaller the time constant τ2, the faster the avoiding velocity. The size and location of the determination area may be determined as the avoidance condition.

In step S16, both the tracking control (S18) and the avoidance control (S20) may be executed. In this case, one of the control operations may be executed preferentially in a state where the other control is executable.

Specific examples of the tracking control and the avoidance control will be described respectively.

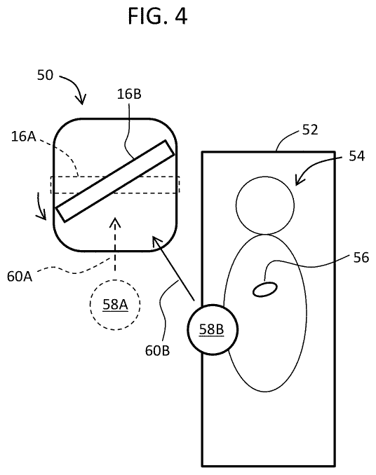

FIG. 4 illustrates an example of the tracking control. A bed 52 is placed adjacent to an ultrasonic diagnostic apparatus 50. A subject 54 is lying on the bed 52. When the display is in its original attitude 16A, the head 58A of an operator is located in front of the display 16A. The direction of the operator's line of sight at this time is indicated with an arrow 60A. The position of the operator's head continuously changes with the contact of a probe 56 to the subject 54 or with the contacting location of the probe 56. The operator's head after the position change is indicated by reference numeral 58B, and the direction of the operator's line of sight at this time is indicated by an arrow 60B. The attitude of the display is changed as indicated by reference numeral 16B with the change in the position of the operator's head. It is thus possible to keep the screen of the display 16B automatically facing the operator while the operator is viewing the ultrasound image to thereby help the operator view the ultrasound image easily.

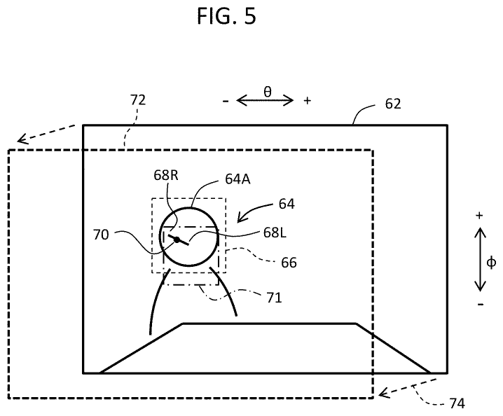

FIG. 5 illustrates an example imaging analyzing method for the tracking control. A camera image 62 includes an operator image 64 including a head image or a face image 64A. To extract the head image 64A, a region of interest 66 is automatically fixed. The head image or an area where the head image is present may be specified with an image recognition technique.

The head image 64A is used to specify a representative coordinate 70 included in the head image 64A. The representative coordinate 70 may be determined as an intermediate point between locations of two eyes 68R and 68L that are specified. The representative coordinate may be other locations including, for example, a location of the center of gravity of the head image 64A or a location of the midpoint of the head image 64A.

The camera image 62 includes a target area or target position that is not shown, in the center. When the representative coordinate is outside of the target area, the orientation or attitude of the display is changed such that the representative coordinate is present within the target area (see reference numeral 74). To change the orientation of the display, a vector directing from the representative coordinate toward the center point of the target area may be calculated. Then, based on two components that define the vector, the direction and velocity for changing the turning angle θ and the tilt angle φ may be determined. At this time, the position of the display, in addition to the orientation, may be changed.

In the example illustrated in FIG. 5, a camera image 72 after change of the orientation is acquired. The camera image 72 includes, in the center portion, a target area 71 which includes the representative coordinate 70. In practice, the tracking control including image analysis is executed for each frame, and the orientation of the display continuously changes with the movement of the head. Here, the moving velocity at this time depends on the time constant τ1.

The orientation of the face may be determined from the location of the representative coordinate in the head image or in the face image 64A. The tracking control may be executed only when the operator is facing the display. The orientation of the face may be determined by other methods including detection of a vector of the line of sight.

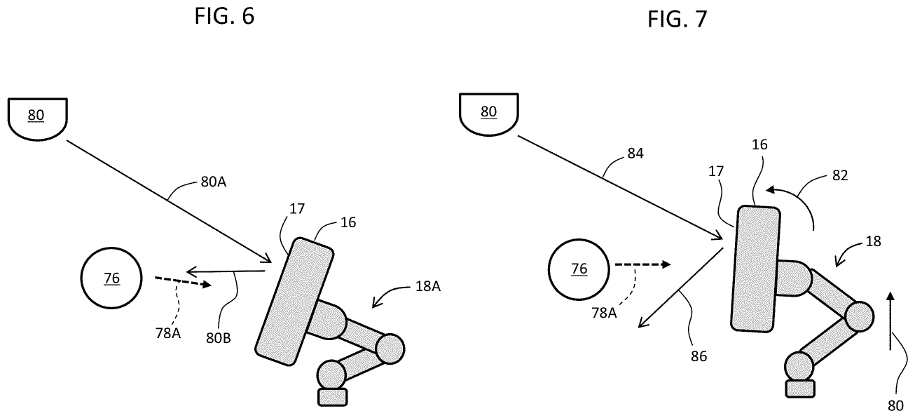

FIG. 6 illustrates an illumination reflection state. The display 16 is held by the support mechanism 18A. The screen 17 of the display 16 faces upward. The operator's head is indicated with reference numeral 76 and the line of sight of the operator is indicated with an arrow 78A. A light 80 is disposed on the ceiling of a laboratory. A ray of light 80A from the light 80 is reflected on the screen 17 to generate a ray of light 80B, which enters the operator's viewing field. In other words, an illumination image is reflected within the screen 17, which disturbs observation of an ultrasound image. In a laboratory which is typically a dark room but where light is not completely turned off, reflection of an illumination image may occur depending on the conditions.

The ultrasonic diagnostic apparatus according to the embodiment executes the avoidance control as illustrated in FIG. 7 in response to determination of presence of illumination reflection. More specifically, the driving source of the support mechanism 18 is controlled to raise the display 16 (see arrow 80) and simultaneously change the tilt angle of the display 16 so that the screen 17 is oriented further downward (see arrow 82). This control avoids a ray of light 86 generated by reflection of a ray of light 84 from the light 80 on the screen 17 from entering the viewing field of the operator (see reference numeral 78A). The issue of the illumination reflection can also be alleviated even if the ray of light 86 enters the viewing field of the operator. After the avoidance control, the position and attitude of the display are determined to allow the screen to be opposite the operator's head.

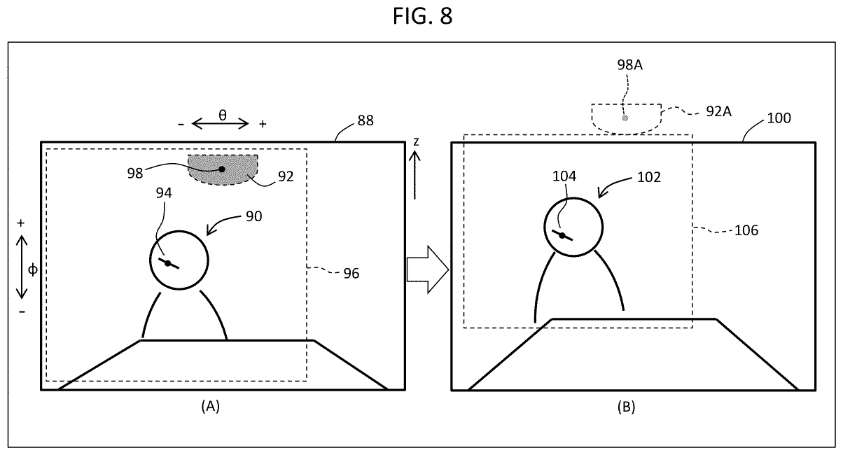

FIG. 8 illustrates an example image analyzing method for the avoidance control. (A) denotes a state before the avoidance control and (B) denotes a state after the avoidance control. As denoted by (A), a camera image 88 includes an operator image 90. It is possible to determine, based on the location of a representative coordinate 94 in a head image, that the operator is facing the display. An illumination image 92 is reflected on the camera image 88. It is possible to substantially determine whether reflection is present on the screen from the overlapping relationship between the observation field of view and the imaging field of view, based on the location of the illumination image 92 within the camera image 88 (actually based on a spatial relationship between the operator and the display).

In the camera image 88, a determination area 96 is set to determine the presence of reflection, for example. In determining the determination area 96, the head image or the representative coordinate 94 may be used as a reference. When the determination area 96 includes an illumination image or a contour of the illumination image, presence of reflection is determined. The presence of reflection may also be determined according to the presence of the representative coordinate (that is a center location, for example) of the illumination image 92 within the determination area 96.

In the embodiment, in response to the determination of presence of reflection, the display is raised or the imaging field of view is lifted upward along the z direction, and the tilt angle φ is changed toward the negative direction or the imaging field of view is changed downward. The optimal avoidance pattern may be automatically selected as appropriate from among a plurality of avoidance patterns that are previously registered.

As denoted by (B), in a camera image 100 that is modified, an illumination image 92A as well as its representative coordinate 98A are outside and above a determination area 106. In the illustrated example, the determination area 106 is set with reference to a representative coordinate 104 of an operator image 102 within the camera image 100. The determination area may be defined to have the representative coordinate 104 as its center.

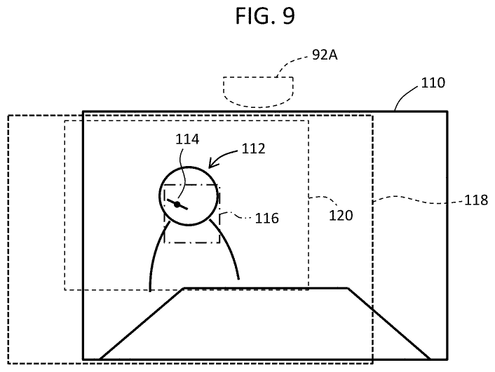

FIG. 9 illustrates a result of the tracking control and the avoidance control executed simultaneously. The position and attitude of the display are controlled to remove the illumination image 92A outside of the determination area that is set for a camera image 110. However, a representative coordinate 114 of an operator image 112 is significantly deviated from the center of the camera image 110. Under the tracking control, the display is turned and a camera image 118 is acquired. A target area 116 including the representative coordinate 114 is then defined with respect to the camera image 118. In a new determination area 120 that is set within the camera image 118, the illumination image 92A is not reflected. In other words, the display screen is oriented to face the operator while avoiding illumination reflection. When it is not possible to perform the two control operations simultaneously, a predetermined one of the control operations may be performed first. Alternatively, the two control operations may be performed in various manners.

According to the embodiment described above, an image captured by a camera fixed to the display is analyzed to perform the tracking control and the avoidance control. No complicated configuration is therefore necessary to achieve these control operations. The support mechanism may include limiters for the respective movable portions to stop the movement of the movable portions in response to generation of a predetermined amount of load or greater load.

1. An ultrasonic diagnostic apparatus comprising:
a display configured to display an ultrasound image;
a camera disposed on the display or adjacent to the display to capture an image of a front space including an operator and form a camera image;
a support mechanism supporting the display, the support mechanism having a driving source configured to change at least one of a position and attitude of the display, and
a controller configured to control the driving source based on an operator image included in the camera image.

2. The ultrasonic diagnostic apparatus according to claim 1, wherein
the camera is fixed to the display, and
an imaging field of view of the camera and an observation field of view of the display overlap with each other.

3. The ultrasonic diagnostic apparatus according to claim 2, wherein
the controller determines a representative position of the operator image in the camera image to change at least one of the position and attitude of the display based on the representative position.

4. The ultrasonic diagnostic apparatus according to claim 3, wherein
the representative position is determined from a head image of the operator image, and
the controller controls at least one of the position and attitude of the display to align the representative position with or near a target position in the camera image.

5. The ultrasonic diagnostic apparatus according to claim 1, wherein
the controller determines whether the operator faces the display based on the camera image and controls the driving source when the operator faces the display.

6. The ultrasonic diagnostic apparatus according to claim 1, wherein
the controller determines a moving velocity to change at least one of the position and attitude of the display in accordance with a designated response condition.

7. The ultrasonic diagnostic apparatus according to claim 1, wherein
the controller determines whether illumination reflection is present on a screen of the display based on an illumination image included in the camera image, and, in response to determination of the presence of the illumination reflection, changes at least one of the position and attitude of the display to reduce or eliminate the illumination reflection.

8. The ultrasonic diagnostic apparatus according to claim 7, wherein
the controller determines the presence of the illumination reflection when a contour or a representative coordinate of the illumination image is within a determination area of the camera image, and changes at least one of the position and attitude of the display to remove the contour or the representative coordinate of the illumination image outside of the determination area.
